(19) United States
(12) Patent Application Publication
(10) Pub. No.: US 2026/0076738 A1
(43) Pub. Date: Mar. 19, 2026

(54) STAGGERED PAIRS OF BIASED ABLATION ELECTRODES ON BASKET CATHETER

(71) Applicant: Biosense Webster (Israel) Ltd., Yokneam (IL)

(72) Inventors: Assaf GOVARI, Haifa (IL); Christopher Thomas BEECKLER, Brea, CA (US); Joseph Thomas KEYES, Sierra Madre, CA (US); Justin George LICHTER, Irvine, CA (US)

(73) Assignee: Biosense Webster (Israel) Ltd., Yokneam (IL)

(21) Appl. No.: 19/399,401

(22) Filed: Nov. 24, 2025

Related U.S. Application Data
(63) Continuation of application No. 17/472,487, filed on Sep. 10, 2021, now Pat. No. 12,478,424.

(51) Int. Cl. A61B 18/14; A61B 18/00

(52) U.S. Cl. CPC A61B 18/1492; A61B 2018/0016; A61B 2018/00214; A61B 2018/00267; A61B 2018/00577; A61B 2018/00613; A61B 2018/1417; A61B 2018/1475

(57) ABSTRACT

Embodiments of the present invention include a medical probe having a flexible insertion tube, a basket assembly, and a plurality of electrodes. The basket assembly has a proximal end that is connected distally to the insertion tube and includes a plurality of resilient spines, which have respective outer sides and inner sides and are configured to bow radially outward from an axis of the basket assembly and are conjoined at the proximal end and at a distal end of the basket assembly. The plurality of electrodes are fixed to respective ones of the spines in respective longitudinal positions that are staggered on any given spine relative to the longitudinal positions of the electrodes on the spines adjacent to the given spine, and which include a conductive material that is biased towards the respective outer sides of the spines.

### CROSS-REFERENCE TO RELATED APPLICATIONS

This application is a continuation of prior filed U.S. patent application Ser. No. 17/472,487 filed Sep. 10, 2021 (Attorney Ref. No.: BIO6462USNP1-253757.000218). The entire contents of which are hereby incorporated by reference.

### FIELD OF THE INVENTION

The present invention relates generally to medical probes, and specifically to a medical probe comprising a basket assembly comprising spines having staggered pairs of biased ablation electrodes.

### BACKGROUND OF THE INVENTION

Arrhythmias are abnormal heart rhythms that are typically caused by a small area of cardiac tissue that produces irregular heartbeats. Cardiac ablation is a medical procedure that can be performed to treat an arrhythmia by destroying the area of the cardiac tissue causing the irregular heartbeats. Some medical systems use irreversible electroporation (IRE) to ablate cardiac tissue. IRE is a nonthermal ablation method based on the unrecoverable permeabilization of cell membranes caused by short pulses of high voltage delivered to the tissue.

U.S. Patent Application 2011/0118726 to de la Rama et al., describes an ablation catheter comprising a staggered ablation element assembly. The assembly may be expanded and collapsed, and may comprise ablation elements that are discretely spaced from each other longitudinally and/or laterally, wherein at least two of the ablation elements are spaced from one another longitudinally.

U.S. Patent Application 2015/0282859 to Bencini et al., describes a balloon catheter with flexible electrode assemblies. The electrodes in assembly may be staggered, rather than having a regular spacing along longitudinal lines. Such a staggered arrangement can be particularly advantageous with electrodes have a three-dimensional structure, to prevent the electrodes from bunching up when the balloon is in a collapsed arrangement.

U.S. Patent Application 2015/0366508 to Chou et al., describes an expandable catheter assembly. The assembly comprises spines having linearly staggered connection portions. These portions allow an efficiently radially compact design of a surrounding shaft and allow efficient radially compacting of the spines due to the staggered positioning.

U.S. Pat. No. 9,848,795 to Marecki et al., describes an electrode assembly for use in a cardiac mapping procedure. The assembly may comprise bipolar pairs of electrodes having a staggered configuration along the length of a flexible member or a spine.

U.S. Pat. No. 5,782,239 to Webster describes electrode configurations for cardiovascular mapping catheters. The distal end of the catheter comprises a plurality of arms that carry electrodes. The arms bow outwardly upon extension of the inner catheter from the outer catheter to form a three-dimensional shape. Each arm has a spine of a superelastic material. Electrode pairs on each arm are able to be spaced evenly with respect to each other and can have staggered spacing with respect to the electrodes on other arms.

U.S. Pat. No. 10,617,467 to Viswanathan et al., describes devices for focal ablation. The ablation device includes a set of spines coupled to a catheter for medical ablation therapy, and each spine of the set of spines may include a set of electrodes formed on that spine. In some embodiments, the ablation device includes a first spine and a second spine adjacent to the first spine, wherein an electrode of the first spine is disposed closer to a distal end of the ablation device relative to an electrode of the second spine.

U.S. Pat. No. 8,295,902 to Salahieh et al., describes a low-profile electrode assembly. In one embodiment, the assembly may comprise rows of electrodes that line up or can be staggered. IN another embodiment, the assembly may comprise staggered conductive pads 59 that can aid in providing a low profile to reduce a stack-up of the regions of enlarged width or diameter substrate.

The description above is presented as a general overview of related art in this field and should not be construed as an admission that any of the information it contains constitutes prior art against the present patent application.

### SUMMARY OF THE INVENTION

There is provided, in accordance with an embodiment of the present invention, a medical probe, including a flexible insertion tube, a basket assembly having a proximal end that is connected distally to the insertion tube and including a plurality of resilient spines, which have respective outer sides and inner sides and are configured to bow radially outward from an axis of the basket assembly and are conjoined at the proximal end and at a distal end of the basket assembly, and a plurality of electrodes, which are fixed to respective ones of the spines in respective longitudinal positions that are staggered on any given spine relative to the longitudinal positions of the electrodes on the spines adjacent to the given spine, and which include a conductive material that is biased towards the respective outer sides of the spines.

In one embodiment, the medical probe further includes an electric signal generator coupled to the electrodes. In some embodiments, the electrical signal generator is configured to simultaneously deliver irreversible electroporation (IRE) pulses between one or more of the electrodes. In other embodiments, the one or more of the electrodes include at least two electrodes fixed to a single given spine or fixed to multiple spines. In additional embodiments, the electrical signal generator is configured to deliver radio frequency energy to one or more of the electrodes.

In another embodiment, the medical probe further includes an insertion sheath containing the insertion tube, the insertion tube has a longitudinal axis, and there is a specified maximum number of the electrodes at any location on the longitudinal axis when the basket assembly is disposed within the insertion sheath and assumes a collapsed configuration. Some embodiments, the electrodes are dimensioned and positioned on the spines so as to fit within the insertion sheath when the basket assembly assumes the collapsed configuration.

In an additional embodiment, the basket assembly has a longitudinal axis and an equator, the longitudinal axis joins the proximal and the distal ends of the basket assembly, the equator corresponding to a line through the spines on a plane orthogonal to the axis and through a midpoint between a proximal end of the basket assembly and a distal end of the assembly on the longitudinal axis, and each of the spines includes at least one electrode on each side of the equator.

In a further embodiment, any pair of alternating spines in the basket assembly includes first and second spines has a first electrode at a longitudinal position fixed to the first the alternating spine that overlaps the longitudinal position of a second electrode fixed to the second spine.

In a supplemental embodiment, any pair of alternating spines in the basket assembly includes first and second spines having a pair of electrodes including a first electrode fixed to the first spine and a second electrode fixed to the second spine, the first and the second electrodes are fixed to their respective spines at a specified longitudinal distance to the distal end of the basket assembly.

In another embodiment, any pair of adjacent spines in the basket assembly includes first and second spines having a pair of electrodes including a first electrode fixed to the first spine and a second electrode fixed to the second spine, and the staggering includes the first and the second electrodes fixed to their respective spines at a specified longitudinal distance to each other. In some embodiments, each of the electrodes includes a length on the axis of the basket assembly, and the specified longitudinal distance is greater than the length of the electrodes.

There is also provided, in accordance with an embodiment of the present invention, a method for fabricating a medical probe, including providing a flexible insertion tube, providing a basket assembly having a proximal end that is connected distally to the insertion tube and including a plurality of resilient spines, which have respective outer sides and inner sides and are configured to bow radially outward from an axis of the basket assembly and are conjoined at the proximal end and at a distal end of the basket assembly, and providing a plurality of electrodes, which are fixed to respective ones of the spines in respective longitudinal positions that are staggered on any given spine relative to the longitudinal positions of the electrodes on the spines adjacent to the given spine, and which include a conductive material that is biased towards the respective outer sides of the spines.

There is provided, in accordance with an embodiment of the present invention, a method for treatment, including inserting, into a body cavity, an insertion tube having a distal end containing a lumen passing through the insertion tube, deploying, into the body cavity from the distal end, a basket assembly having a proximal end that is connected distally to the insertion tube and including a plurality of resilient spines, which have respective outer sides and inner sides and are configured to bow radially outward from an axis of the basket assembly and are conjoined at the proximal end and at a distal end of the basket assembly, and a plurality of electrodes, which are fixed to respective ones of the spines in respective longitudinal positions that are staggered on any given spine relative to the longitudinal positions of the electrodes on the spines adjacent to the given spine, and which include a conductive material that is biased towards the respective outer sides of the spines, positioning the basket assembly so that one or more of the electrodes press against tissue in the body cavity, selecting at least two sets of the electrodes, and conveying, between the selected two sets of the electrodes, ablation energy to the tissue.

### BRIEF DESCRIPTION OF THE DRAWINGS

The disclosure is herein described, by way of example only, with reference to the accompanying drawings, wherein:

FIG. 1 is a schematic pictorial illustration of a medical system comprising a basket assembly, in accordance with an embodiment of the present invention;

FIG. 2 is a schematic illustration of the basket assembly in an expanded configuration, in accordance with an embodiment of the present invention;

FIG. 3 is a schematic illustration showing staggered electrodes fixed to spines of the basket assembly, in accordance with an embodiment of the present invention;

FIG. 4 is a schematic side view of the basket assembly in a collapsed configuration, in accordance with an embodiment of the present invention;

FIG. 5 is a flow diagram that schematically illustrates a method of using the medical probe to perform a tissue ablation medical procedure in a chamber of a heart, in accordance with an embodiment of the present invention; and

FIGS. 6 and 7 are a schematic pictorial illustrations of the basket assembly inside the chamber of the heart during the medical procedure, in accordance with an embodiment of the present invention.

### DETAILED DESCRIPTION OF EMBODIMENTS

### Overview

When delivering pulsed field ablation (PFA) in an IRE (irreversible electroporation) procedure, electrodes should contact the tissue being ablated with as large a surface area as possible. Embodiments of the present invention provide a medical probe comprising an insertion tube configured for insertion into a body cavity of a patient. The medical probe also comprises a basket assembly having a proximal end that is connected distally to the insertion tube, i.e., is connected to a distal end of the insertion tube. The basket assembly comprises a plurality of resilient spines. In some embodiments, the spines have respective outer sides and inner sides and are configured to bow radially outward from an axis of the basket assembly and are conjoined at the proximal end and at a distal end of the basket assembly.

The medical probe additionally comprises a plurality of electrodes, which are fixed to respective ones of the spines in respective longitudinal positions that are staggered on any given spine relative to the longitudinal positions of the electrodes on the spines adjacent to the given spine. The electrodes comprise a conductive material that is biased towards the respective outer sides of the spines.

Medical systems implementing embodiments of the present invention enable an IRE ablation field to be generated between two selected sets of electrodes, each set comprising one or more individual electrodes, and a full IRE ablation typically involves generating multiple fields between multiple sets. By having a at least two electrodes on each spine, the number of possible electrode selections is greatly increased.

In some embodiments, the medical probe comprises an insertion tube, the basket assembly assumes a collapsed configuration when positioned within the insertion tube, and the basket assembly can assume an expanded configuration when deployed from a distal end of the insertion tube to a body cavity. By staggering the biased electrodes on spines of the basket assembly, medical probes implementing embodiments of the present invention may require less space (i.e., diameter) when the basket assumes the collapsed configuration within the insertion tube.

### System Description

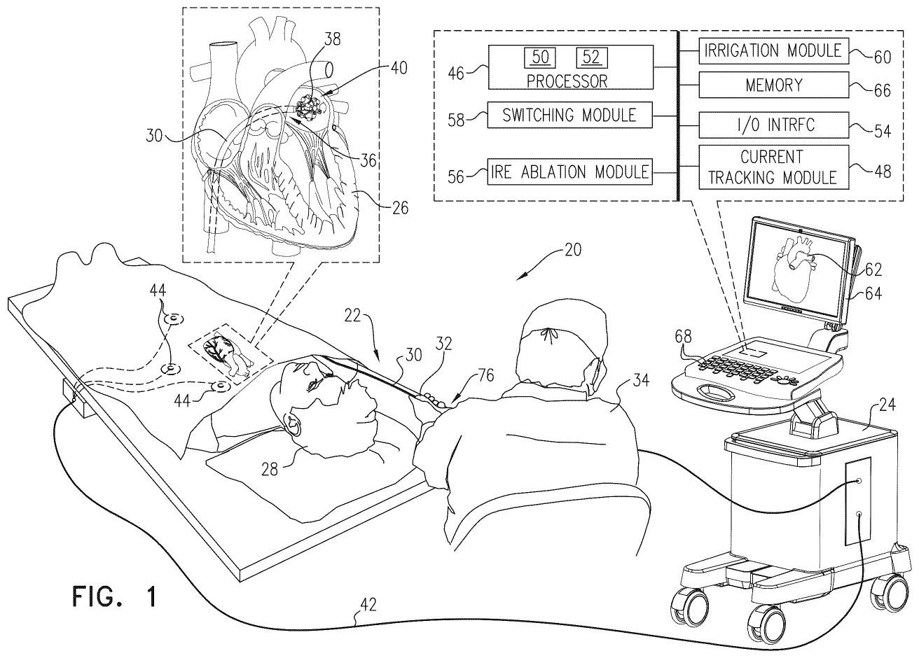

FIG. 1 is a schematic, pictorial illustration of a medical system 20 comprising a medical probe 22 and a control console 24, in accordance with an embodiment of the present invention. Medical system 20 may be based, for example, on the CARTO® system, produced by Biosense Webster Inc. of 31 Technology Drive, Suite 200, Irvine, CA 92618 USA. In embodiments described hereinbelow, medical probe 22 can be used for diagnostic or therapeutic treatment, such as for performing ablation procedures in a heart 26 of a patient 28. Alternatively, medical probe 22 may be used, mutatis mutandis, for other therapeutic and/or diagnostic purposes in the heart or in other body organs.

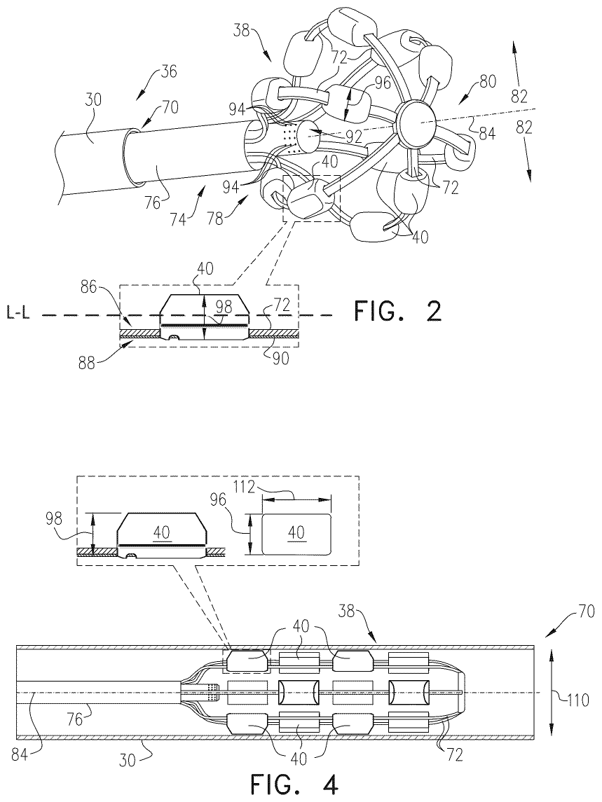

Probe 22 comprises a flexible insertion sheath 30 and a handle 32 coupled to a proximal end of the insertion sheath. Probe 22 also comprises a flexible insertion tube 76 that is contained within insertion sheath 30. During a medical procedure, a medical professional 34 can insert probe 22 through the vascular system of patient 28 so that a distal end 36 of insertion sheath 30 enters a body cavity such as a chamber of heart 26. Upon distal end 36 entering the chamber of heart 26, medical professional 34 can deploy a basket assembly 38 affixed to a distal end 74 of insertion tube 76 (as shown in FIG. 2. Basket assembly 38 comprises a set of electrodes 40, as described in the description referencing FIG. 2 hereinbelow.

To start performing a medical procedure such as irreversible electroporation (IRE) ablation, medical professional 34 can manipulate handle 32 to position distal end 74 so that one or more electrodes 40 engage cardiac tissue at a desired location or locations.

In the configuration shown in FIG. 1, control console 24 is connected, by a cable 42, to body surface electrodes, which typically comprise adhesive skin patches 44 that are affixed to patient 28. Control console 24 comprises a processor 46 that, in conjunction with a current tracking module 48, determines location coordinates of distal end 74 inside heart 26 based on impedances and/or currents measured between adhesive skin patches 44 and electrodes 40 that are affixed to basket assembly 38. In addition to being used as location sensors during a medical procedure, electrodes 40 may perform other tasks such as ablating tissue in the heart.

As described hereinabove, in conjunction with current tracking module 48, processor 46 may determine location coordinates of distal end 74 inside heart 26 based on impedances and/or currents measured between adhesive skin patches 44 and electrodes 40. Such a determination is typically after a calibration process relating the impedances or currents to known locations of distal end 74 has been performed. While embodiments presented herein describe electrodes 40 that are (also) configured to deliver IRE ablation energy to tissue in heart 26, configuring electrodes 40 to deliver any other type of ablation energy to tissue in any body cavity is considered to be within the spirit and scope of the present invention.

Processor 46 may comprise real-time noise reduction circuitry 50 typically configured as a field programmable gate array (FPGA), followed by an analog-to-digital (A/D) signal conversion integrated circuit 52. The processor can be programmed to perform one or more algorithms disclosed herein, each of the one or more algorithms comprising steps described hereinbelow. The processor uses circuitry 50 and circuit 52 as well as features of modules which are described in more detail below, in order to perform the one or more algorithms.

Although the medical system shown in FIG. 1 uses impedance or current-based sensing to measure a location of distal end 74, other location tracking techniques may be used (e.g., techniques using magnetic-based sensors). Impedance and current-based location tracking techniques are described, for example, in U.S. Pat. Nos. 5,983,126, 6,456,864 and 5,944,022. The methods of location sensing described hereinabove are implemented in the above-mentioned CARTO® system and are described in detail in the patents cited above.

Control console 24 also comprises an input/output (I/O) communications interface 54 that enables control console 24 to transfer signals from, and/or transfer signals to electrodes 40 and adhesive skin patches 44. In the configuration shown in FIG. 1, control console 24 additionally comprises an electric signal generator 56 and a switching module 58. While embodiments described herein present the electric signal generator as IRE ablation module 56 (i.e., electric signal generator 56 is also referred to herein as IRE ablation module 56), other types of electric signal generators are considered to be within the spirit and scope of the present invention. For example, electric signal generator 56 may be configured to generate radio frequency (RF) energy.

IRE ablation module 56 is configured to generate IRE pulses comprising peak power in the range of tens of kilowatts. As described hereinbelow, medical system 20 performs IRE ablation by delivering IRE pulses between sets of electrodes 40 simultaneously. In some embodiments, a given set of the electrodes comprises two or more of electrodes 40. Using switching module 58, IRE ablation module 56 can deliver one or more IRE pulses independently between each of the sets of the electrodes. The ablation may comprise multiple pulses to different sets, where each set may be comprised of different electrodes for subsequent pulses.

In order to dissipate the minimal amount of heat generated by the pulse and to improve the efficiency of the ablation process, system 20 supplies irrigation fluid (e.g., a normal saline solution) to distal end 74 via a channel (not shown) in insertion tube 30. Control console 24 comprises an irrigation module 60 to monitor and control irrigation parameters, such as the pressure and the temperature of the irrigation fluid.

Typically, based on signals received from electrodes 40 and/or adhesive skin patches 44, processor 46 can generate an electroanatomical map 62 that shows the location of distal end 74 in the patient's body. During the procedure, processor 46 can present map 62 to medical professional 34 on a display 64, and store data representing the electroanatomical map in a memory 66. Memory 66 may comprise any suitable volatile and/or non-volatile memory, such as random-access memory or a hard disk drive.

In some embodiments, medical professional 34 can manipulate map 62 using one or more input devices 68. In alternative embodiments, display 64 may comprise a touchscreen that can be configured to accept inputs from medical professional 34, in addition to presenting map 62.

FIG. 2 is a schematic illustration of distal end 74 comprising basket assembly 38 in an expanded configuration, in accordance with an embodiment of the present invention. Basket assembly 38 can assume an expanded configuration when unconstrained, such as by being advanced out of an insertion sheath lumen 70 of sheath 30.

By way of example, basket assembly 38 comprises a plurality of resilient spines 72 that are formed at a distal end 74 of insertion tube 76. A proximal end 78 of basket assembly 38 is connected to distal end 74 of insertion tube 76, and spines 72 are cojoined at a distal end 80 of the basket assembly.

During a medical procedure, medical professional 34 can deploy basket assembly 38 by extending insertion tube 76 from sheath 30. Spines 72 may have oval (e.g., elliptical or circular) or rectangular (that may appear to be flat) cross-sections, and typically comprise a flexible, resilient material (e.g., a shape-memory alloy such nickel-titanium, also known as Nitinol). In its expanded (i.e., unconstrained) configuration, basket assembly 38 has an expanded arrangement wherein spines 72 bow in a radially outward direction 82 from a longitudinal axis 84 of the basket assembly that joins proximal end 78 and distal end 80 (i.e., of the basket assembly).

In the configuration shown in FIG. 2, one or more electrodes 40 are threaded on to each given spine 72 so as to fit the electrodes to the spines. Each spine 72 has an outer side 86 and an inner side 88. In embodiments of the present invention, for a given electrode 40 is fitted to a given spine 72, the given spine is assumed to be planar at the given electrode, wherein the plane divides the given electrode asymmetrically so that there is more conductive material on the outer side (of the plane) than on the inner side. The inset of FIG. 2 shows a side view of electrode 40B, the spine 72 extends completely through the electrode 40B on one side such that the spine 72 is offset or “asymmetric” with respect to the center line L-L by being on one side of center line L-L. With the spine 72 offset (by being on one side of center line L-L), electrode 40B will have more of its upper surface (inset of FIG. 2) extending into biological tissues.

In these embodiments, each electrode 40 (i.e., when fitted to a given spine 72) comprises a conductive material that is geometrically biased towards the outer sides of its respective spine, because of the asymmetry referred to above. Therefore, each given electrode 40 has a greater surface area on its outer side compared to the surface area of the given electrode on its inner side. By biasing electrodes 40 to outer side 86 by this asymmetric arrangement, the electrodes deliver more ablation energy from the portion of the electrodes outer side of the spines (i.e., significantly more than the ablation energy delivered from the portion of the electrodes on the inner side of the spines).

As shown in FIG. 4, a plurality of electrodes 40 are provided in which a spine 72 is configured to extend entirely through the electrodes 40 (electrode 40 having a through passage for insertion of the spine shown in the inset) and then electrodes 40 are fixed (e.g., solder or adhesive) to respective ones of the spines in respective longitudinal positions that are staggered on any given spine relative to the longitudinal positions of the electrodes on the spines adjacent to the given spine

In embodiments described herein, electrodes 40 can be configured to deliver ablation energy to tissue in heart 26. In addition to using electrodes 40 to deliver ablation energy, the electrodes can also be used to determine the location of basket assembly 38 and/or to measure a physiological property such as local surface electrical potentials at respective locations on tissue in heart 26.

Examples of materials ideally suited for forming electrodes 40 include gold, platinum and palladium (and their respective alloys). These materials also have very high thermal conductivity which allows the minimal heat generated on the tissue (i.e., by the ablation energy delivered to the tissue) to be conducted through the electrodes to the back side of the electrodes (i.e., the portions of the electrodes on the inner sides of the spines), and then to the blood pool in heart 26.

Probe 22 also comprises a set of wires 90 that couple IRE ablation module 56 to electrodes 40. In some embodiments each spine 72 comprises at least one wire 90 affixed to its inner side 88.

In some embodiments, medical probe 22 comprises a stem 92 that extends longitudinally from the distal end of insertion tube 30. As described supra, control console 24 comprises irrigation module 60 that delivers irrigation fluid to distal end 74, and so to stem 92. Stem 92 comprises multiple spray ports 94, wherein each given spray port 94 is angled to aim delivery of the irrigation fluid to either a given electrode 40 or to tissue in heart 26 (i.e., by aiming the delivery between two adjacent spines 72).

Since electrodes 40 do not comprise spray ports that deliver irrigation fluid, the configuration described hereinabove enables heat to be transferred from the tissue (i.e., during an ablation procedure) to the portion of the electrodes on the inner side of the spines, and the electrodes can be cooled by aiming the irrigation fluid, via spray ports 94, at the portion of the electrodes on the inner side of the spines.

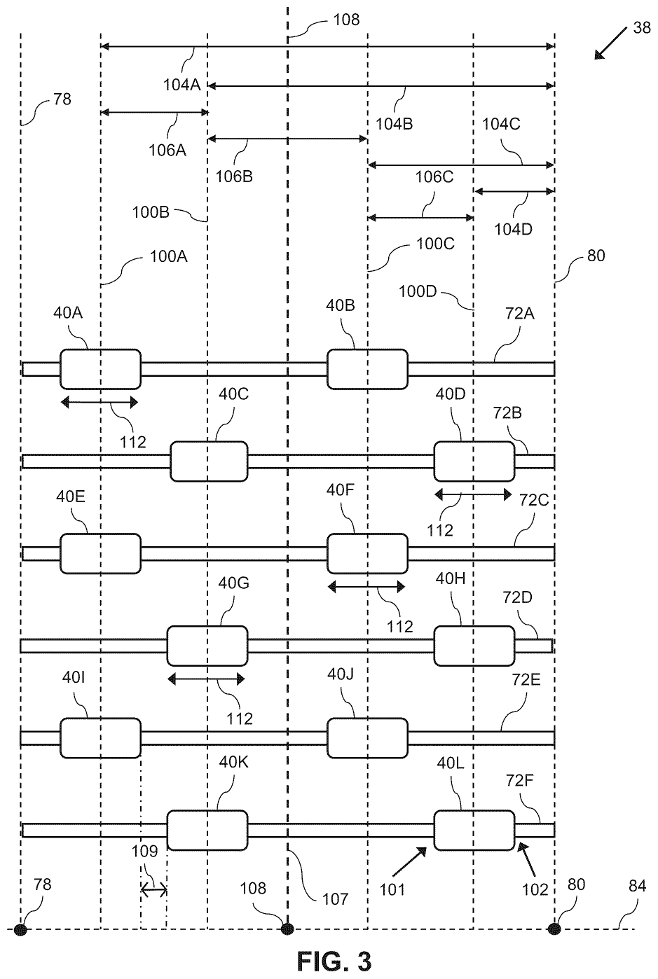

FIG. 3 is a schematic illustration of electrodes 40 fixed to respective ones of spines 72 at respective longitudinal positions 100 (e.g., relative to axis 84), in accordance with an embodiment of the present invention. In embodiments described herein, a given position 100 for a given electrode 40 comprises any latitudinal coordinates along axis 84 encompassed by the given electrode, when the electrode is projected orthogonally onto the axis. For purposes of visual simplicity and clarity, FIG. 3 shows spines 72 in a two-dimensional configuration and positioned parallel to one another.

In FIG. 3, electrodes 40, spines 72 and positions 100 can be differentiated by appending a letter to the identifying numeral, so that the electrodes comprise electrodes 40A-40L, the spines comprise spines 72A-72F and the longitudinal positions comprise positions 100A-100D. While the configuration in FIGS. 2 and 3 present basket assembly 38 comprising six spines 72 with two electrodes 40 affixed to each of the spines, basket assemblies having any plurality of spines 72 with any number of electrodes 40 affixed to each of the spines are considered to be within the spirit and scope of the present invention.

In the configuration shown in FIG. 3:
Spine 72A is adjacent to spines 72F and 72B (i.e., in basket assembly 38), and comprises electrodes 40A and 40B fixed to spine 72A at respective positions 100A and 100C.
Spine 72B is adjacent to spines 72C and 72A (i.e., in basket assembly 38), and comprises electrodes 40C and 40D fixed to spine 72B at respective positions 100B and 100D.
Spine 72C is adjacent to spines 72B and 72D (i.e., in basket assembly 38), and comprises electrodes 40E and 40F fixed to spine 72C at respective positions 100A and 100C.
Spine 72D is adjacent to spines 72C and 72E (i.e., in basket assembly 38), and comprises electrodes 40G and 40H fixed to spine 72D at respective positions 100B and 100D.
Spine 72E is adjacent to spines 72D and 72F (i.e., in basket assembly 38), and comprises electrodes 40I and 40J fixed to spine 72E at respective positions 100A and 100C.
Spine 72F is adjacent to spines 72E and 72A (i.e., in basket assembly 38), and comprises electrodes 40K and 40L fixed to spine 72F at respective positions 100B and 100D.

Therefore, there are no electrodes 40 on adjacent spines 72 that have respective overlapping positions 100. However, pairs of alternating spines (i.e., spines 72A and 72C, spines 72B and 72D, spines 72C and 72E, spines 72D and 72F and spines 72E and 72A) can have electrodes 40 that have respective overlapping positions 100. In other words, adjacent spines 72 have electrodes 40 at staggered positions 100, and alternating spines have electrodes 40 at overlapping positions 100.

In embodiments herein a pair of alternating spines 72 comprises two of the spines that have a single spine between them. For example, spines 72A and 72C are a pair of alternating spines, since they are both adjacent to a single spine 72B that is between them in basket assembly 38.

In some embodiments of the present invention, electrodes 40 are staggered on spines 72 so as to limit the number of electrodes 40 in any given position 100 to a specific number. In the configuration shown in FIG. 3, the specific number is three, so that:
Electrodes 40A, 40E and 40I are fixed to spines 72A, 72C, 72E at position 100A.
Electrodes 40C, 40G and 40K are fixed to respective spines 72B, 72D and 72F at position 100B so that electrodes 40C, 40G, 40K are staggered or offset distance 106A (measured from line 100A to line 100B) with respect to electrode 40A, 40E, 40I on spines 72A, 72C, 72E.
Electrodes 40B, 40F and 40J are fixed to spines 72A, 72C, 72E at position 100C so that electrodes 40B, 40F, 40J are staggered or offset distance 106A with respect to electrodes 40C, 40G, 40K on spines 72B, 72D, 72F.
Electrodes 40D, 40H and 40L are fixed to spines 72B, 72D, 72F at position 100D are offset with respect to electrodes 40B, 40F, 40J on spines 72A, 72C, 72E via offset distance 1006C.

The staggering of the electrodes may comprise fixing the respective longitudinal positions of the electrodes on any given spine relative to the longitudinal positions of the electrodes on the spines adjacent to the given spine. In other words, electrodes 40 can be fixed to spines 72 so that the longitudinal position of any given electrode 40 does not have any overlapping coordinates with the respective longitudinal positions of any of the electrodes fixed to the two spines 72 adjacent to the given spine.

In the configuration presented in FIG. 3, the longitudinal positions of a given electrode 40 on a given spine 72 comprises the longitudinal positions, on axis 84, from a proximal end 101 of the given electrode to a distal end 102 of the given electrode, as shown for electrode 40L. The longitudinal position of the given electrode is typically halfway between the proximal and distal ends of the given electrode.

To implement the staggering, the electrodes can be fixed to the spines at predefined distances 104 from distal end 80 (of basket assembly 38), wherein a distance difference 106 of the distance from a given electrode 40 on a given spine to any electrodes 40 on any adjacent spine 72 is a specified amount.

Typically, electrodes 40 have the same or similar lengths. In some embodiments, the staggering can be implemented by fixing each given electrode 40 on a given spine 72 so that the distance (i.e., distance difference 106 as shown in FIG. 3) between the longitudinal position of the given electrode and the longitudinal position of any electrode 40 fixed to an adjacent spine 72 is greater than length 112 of electrodes 40.

In FIG. 3, electrodes 40, distances 104 and distance differences 106 are differentiated by appending a letter to the identifying numeral, so that the distances comprise distances 104A-104D and the differences comprise distance differences 106A-106D. These distances and differences can be measured along axis 84. In the example shown in FIG. 3:
Electrodes 40A, 40E and 40I have distance 104A to distal end 80.
Electrodes 40C, 40G and 40K have distance 104B to distal end 80.
Electrodes 40B, 40F and 40J have distance 104C to distal end 80.
Electrodes 40D, 40H and 40L have distance 104D to distal end 80.
The offset distance between the center of electrodes 40A/40E/40I and center of electrodes 40C/40G/40K comprises distance difference 106A. The offset distance 106A can be measured from the corresponding edge of one electrode 40A to the corresponding edge of the offset electrode 40C.
The distance between electrodes 40C/40G/40K and electrodes 40B/40F/40J comprises offset distance difference 106B. The offset distance 106B can be measured from the corresponding edge of one electrode 40C to the corresponding edge of the offset electrode 40F.
The distance between electrodes 40B/40F/40J and electrodes 40D/40H/40L comprises offset distance difference 106C. The offset distance 106C can be measured from the corresponding edge of one electrode 40B to the corresponding edge of the offset electrode 40D.

In some embodiments, electrodes 40 may be distributed evenly on both sides of an equator 107 of basket assembly 38. Equator 107 can be assumed to correspond to a line through the spines on a plane orthogonal to axis 84 and through a midpoint 108 between proximal end 78 and distal end 80.

In the configuration shown in FIGS. 2 and 3, there are two electrodes 40 inserted through by the spine and thereafter fixed to each spine 72, wherein electrodes 49A, 40C, 40E, 40G, 40I and 40K are on the proximal side of equator 107, and electrodes 40B, 40D, 40F, 40H, 40J and 40L are on the distal side of equator 107.

Electrodes 40 have a width 96 and a height 98. The width of a given electrode attached 40 to a given spine 72 is assumed to correspond to the maximum length of a line in the given electrode, where the line resides in a plane parallel to the plane of the given spine where the given electrode is attached, and the line is orthogonal to the direction of the given spine. The height of the given electrode is assumed to correspond to a maximum length of a line in the given electrode, where the line resides in a plane orthogonal to the plane of the given spine.

In some embodiments, spines 72 are inserted through electrodes 40 where each electrode can be slid along the spines 72 and then affixed to spines 72 so that a distance 109 (i.e., along axis 84) between the proximal end of any given electrode 70 on a first spine 72 and the distal end of any electrode 70 on any adjacent spine is at least zero. This distance 109 enables basket assembly 38 to collapse within sheath 30 as described in the description referencing FIG. 4 hereinbelow.

FIG. 4 is a schematic side view of basket assembly 38 in a collapsed configuration and arranged generally along longitudinal axis 84, in accordance with an embodiment of the present invention. As shown in FIG. 4, the outward bias of electrodes 40 enables spines 72 to lay flush with insertion tube 76 when basket assembly 38 is collapsed within sheath 30.

In some embodiments, lumen 70 has a lumen diameter 110 between 3.0 mm and 3.33 mm (e.g., medical probe 22 is a 10 French catheter). In some embodiments, each given electrode 40 can be dimensioned (i.e., width 96, height 98 and length 112 are set) and positioned on their spines so that the electrodes (i.e., when fixed to their respective spines 72) fit into lumen 70 when basket assembly 38 is collapsed.

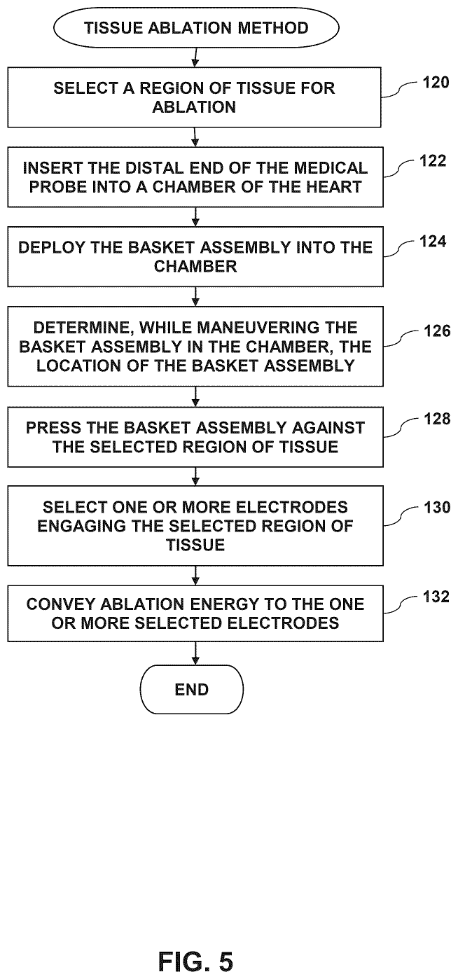

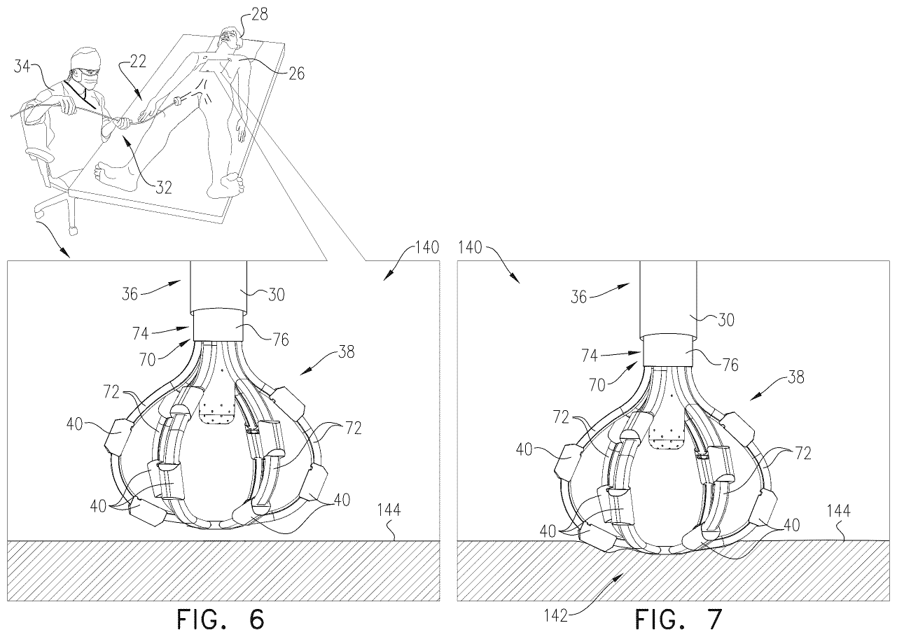

FIG. 5 is a flow diagram that schematically illustrates a method of using medical probe 22 to perform a tissue ablation medical procedure in a chamber 140 of heart 26, and FIGS. 6 and 7 are schematic pictorial illustrations of distal end 74 inside the chamber of the heart during the medical procedure, in accordance with an embodiment of the present invention.

In a tissue selection step 120, medical professional 34 selects a region 142 of intracardial tissue 144, and in an insertion step 122, the medical professional inserts distal end 36 of insertion sheath 30 into chamber 140 of heart 26.

In a deployment step 124, medical professional deploys basket assembly 38 from lumen 70 to chamber 140, as shown in FIG. 6.

As medical professional 34 maneuvers the basket assembly within the chamber (e.g., by manipulating handle 32), processor 46 determines, in a location determination step 126, the location(s) of the basket assembly within the chamber. In some embodiments, processor 46 can present, to medical professional 34 (i.e., in map 62 on display 64), the location(s) of basket assembly 38.

In a positioning step 128, medical professional manipulates handle 32 so as to press one or more electrodes 40 in basket assembly 38 against region 142 of tissue 144, as shown in FIG. 7.

In an electrode selection step 130, medical professional 34 selects (e.g., using a given input device 68) at least one set of electrodes 40 that is engaging intracardiac tissue 144.

In some embodiments, the selected set of electrodes comprise pairs of electrodes 40. In these embodiments, medical system 20 performs IRE ablation by delivering IRE pulses between pairs of electrodes 40. The electrodes in any given set may be fixed to a single spine 72 or to multiple spines 72. Using switching module 58, IRE ablation module 56 can deliver one or more IRE pulses independently between each of the sets of the electrodes.

Finally, in an ablation step 132, in response to an input from medical professional 34, IRE ablation module 56 conveys IRE pulses to the selected electrodes 40A, which the electrodes deliver to the selected region of intracardiac tissue 144, and the method ends. In some embodiments, IRE ablation module 56 can the input from a given input device 68 or from an additional input device (not shown) on handle 32.

While embodiments herein describe IRE ablation module 56 delivering IRE pulses to one or more electrodes 40 so as to ablate intracardiac tissue 144, configuring medical system 20 to deliver, to any electrode 40, other types of ablation energy (e.g., radiofrequency energy) is considered to be within the spirit and scope of the present invention.

It will be appreciated that the embodiments described above are cited by way of example, and that the present invention is not limited to what has been particularly shown and described hereinabove. Rather, the scope of the present invention includes both combinations and subcombinations of the various features described hereinabove, as well as variations and modifications thereof which would occur to persons skilled in the art upon reading the foregoing description and which are not disclosed in the prior art.

1. A medical probe, comprising:
a flexible insertion tube extending along a longitudinal axis;
a basket assembly having a proximal end that is connected distally to the insertion tube and comprising a plurality of spines configured to bow radially outward from the longitudinal axis to define an interior portion of the basket assembly, a first side of each spine of the plurality of spines facing the interior portion; and
a plurality of electrodes disposed on the plurality of spines in respective longitudinal positions that are staggered on any given spine relative to the longitudinal positions of electrodes on an adjacent spine to the given spine, each electrode of the plurality of electrodes defining a through passage extending therethrough that is asymmetrically positioned with respect to an inwardly-facing side and an outwardly-facing side of the electrode, the inwardly-facing side of the electrode facing the interior portion and the outwardly-facing side of the electrode facing away from the interior portion, the through passage configured to receive a spine of the plurality of spines such that the first side of the spine is positioned a first distance from the inwardly-facing side of the electrode and a second side of the spine is positioned a second distance from the outwardly-facing side of the electrode, the first distance being less than the second distance.

2. The medical probe of claim 1, an outwardly-facing side of the electrode being substantially flat.

3. The medical probe of claim 1, the inwardly-facing side of the electrode being substantially flat.

4. The medical probe of claim 1, wherein each electrode of the plurality of electrodes comprises a greater amount of conductive material on the second side of the spine than on the first side of the spine.

5. The medical probe of claim 1, the basket assembly comprising an equator, the equator corresponding to a plane orthogonal to the longitudinal axis and through a midpoint between the proximal end of the basket assembly and a distal end of the basket assembly on the longitudinal axis, and each of the spines includes at least one electrode on each side of the equator.

6. The medical probe of claim 5, the plurality of spines comprising four spines.

7. The medical probe of claim 6, the four spines comprising a first set of spines including a first spine and a third spine, each spine of the first set of spines including a first electrode on a first side of the equator and a second electrode on a second side of the equator, the first electrodes of the first set of spines being aligned along the longitudinal axis and the second electrodes of the first set of spines being aligned along the longitudinal axis.

8. The medical probe of claim 7, comprising a second set of spines, including a second spine adjacent to the first and third spines, a fourth spine adjacent to the first and third spines, the second spine provided on an opposite side of the longitudinal axis from the fourth spine, each spine of the second set of spines including a first electrode on a first side of the equator and a second electrode on a second side of the equator, the first electrodes of the second set of spines being aligned along the longitudinal axis and positioned along the longitudinal axis between the first and second electrodes of the first set of spines, the second electrodes of the second set of spines being aligned along the longitudinal axis, and the second electrodes of the first set of spines being positioned along the longitudinal axis between the first and second electrodes of the second set of spines.

9. The medical probe of claim 8, no portion of the electrodes of the first set of spines overlap with a portion of the electrodes of the second set of spines at any position along the longitudinal axis.

10. The medical probe of claim 5, the plurality of spines comprising six spines.

11. The medical probe of claim 10, the six spines comprising a first set of spines including a first spine, a third spine, and a fifth spine, each spine of the first set of spines including a first electrode on a first side of the equator and a second electrode on a second side of the equator, the first electrodes of the first set of spines being aligned along the longitudinal axis and the second electrodes of the first set of spines being aligned along the longitudinal axis.

12. The medical probe of claim 11, comprising a second set of spines, including a second spine adjacent to the first and third spines, a fourth spine adjacent to the third and fifth spines, and a sixth spine adjacent to the first and fifth spines, each spine of the second set of spines including a first electrode on a first side of the equator and a second electrode on a second side of the equator, the first electrodes of the second set of spines being aligned along the longitudinal axis and positioned along the longitudinal axis between the first and second electrodes of the first set of spines, the second electrodes of the second set of spines being aligned along the longitudinal axis, and the second electrodes of the first set of spines being positioned along the longitudinal axis between the first and second electrodes of the second set of spines.

13. The medical probe of claim 12, no portion of the electrodes of the first set of spines overlap with a portion of the electrodes of the second set of spines at any position along the longitudinal axis.

14. A method for fabricating a medical probe, comprising:
providing a flexible insertion tube extending along a longitudinal axis;
fabricating a basket assembly comprising:
providing a plurality of spines;
providing a plurality of electrodes each electrode of the plurality of electrodes defining a through passage extending therethrough that is asymmetrically positioned with respect to an inwardly-facing side and an outwardly-facing side of the electrode;
threading a first spine of the plurality of spines through the through passages of a first set of at least two electrodes of the plurality of electrodes ;
threading a second spine of a plurality of spines through the through passages of a second set of at least two electrodes of the plurality of electrodes;
fixing the first set of electrodes to the first spine and the second set of electrodes to the second spine such that the plurality of electrodes are staggered in respective longitudinal positions with respect to each other along the first spine and the second spine; and
joining the plurality of spines at their distal ends; and
connecting a proximal end of the plurality of spines to a distal end of the flexible insertion tube.

15. The method of claim 14, the step of fixing the first set of electrodes to the first spine comprising providing at least one electrode on each side of an equator for each spine, the equator corresponding to a midpoint between the proximal end of the basket assembly and the distal end of the basket assembly.

16. The method of claim 15, the step of providing a plurality of spines comprising providing four spines, and the step of fixing the first set of electrodes to the first spine and the second set of electrodes to the second spine further comprises:
longitudinally aligning first electrodes of first set of spines on a first side of the equator, the first set of spines including the first spine and a third spine,
longitudinally aligning second electrodes on a second side of the equator for the first set of spines,
longitudinally aligning first electrodes of a second set of spines on a first side of the equator, the second set of spines including the second spine and a fourth spine, and
longitudinally aligning second electrodes on a second side of the equator for the second set of spines; and
the step of joining the plurality of spines at their distal ends comprises providing the second spine adjacent to the first and third spines, the fourth spine adjacent to the third and first spines and opposite the second spine.

17. The method of claim 16, the step of fixing the first set of electrodes to the first spine and the second set of electrodes to the second spine further comprising positioning the electrodes such that no portion of the electrodes of the first set of spines overlap with a portion of the electrodes of the second set of spines at any position along the longitudinal axis.

18. The method of claim 15, the step of providing a plurality of spines comprising providing six spines, and the step of fixing the first set of electrodes to the first spine and the second set of electrodes to the second spine further comprises:
longitudinally aligning first electrodes of a first set of spines on a first side of the equator, the first set of spines including the first spine, a third spine, and a fifth spine,
longitudinally aligning second electrodes on a second side of the equator for the first set of spines;
longitudinally aligning first electrodes of a second set of spines on a first side of the equator for the second set of spines including the second spine, a fourth spine, and a sixth spine, and
longitudinally aligning second electrodes on a second side of the equator for the second set of spines; and
the step of joining the plurality of spines at their distal ends comprises providing the second spine adjacent to the first and third spines, the fourth spine adjacent to the third and fifth spines, and the sixth spine adjacent to the first and fifth spines.

19. The method of claim 18, the step of fixing the first set of electrodes to the first spine and the second set of electrodes to the second spine further comprising positioning the electrodes such that no portion of the electrodes of the first set of spines overlap with a portion of the electrodes of the second set of spines at any position along the longitudinal axis.

20. The method of claim 14 further comprising providing an electric signal generator coupled to the plurality of electrodes.
